(19) United States
(12) Patent Application Publication
(10) Pub. No.: US 2011/0077680 A1
(43) Pub. Date: Mar. 31, 2011

(54) LARGE MOUTH SNARE DEVICE

(76) Inventor: Richard R. Heuser, Phoenix, AZ (US)

(21) Appl. No.: 12/964,672

(22) Filed: Dec. 9, 2010

Related U.S. Application Data
(63) Continuation of application No. 12/021,204, filed on Jan. 28, 2008.

(51) Int. Cl. A61M 29/00

(52) U.S. Cl. 606/200

(57) ABSTRACT

An apparatus and method for removing an object from a vessel are disclosed. The apparatus includes an expandable catheter, an expandable funnel, and a snare device configured to snare an object within the vessel. A distal end of the expandable catheter may be manipulable such that retraction of the expandable catheter allows the expandable funnel device to expand. Once the snare device has been manipulated to capture the object, the snare device and object may then be retracted into the funnel device, which may then protect the surrounding structures from being damaged by the object. In some embodiments, the expended funnel device, snare device, and object may be retracted into the expandable catheter in order to further protect the surrounding structures from damage.

### CROSS REFERENCE TO RELATED APPLICATIONS

The present application is a Continuation-in-Part of and claims priority to U.S. patent application Ser. No. 12/021,204, filed Jan. 28, 2008, entitled “SNARE DEVICE,” the disclosure of which is hereby incorporated by reference in its entirety.

### TECHNICAL FIELD

Embodiments relate to methods and devices for the removal of objects from within the vasculature, particularly methods and devices for the removal of large and/or sharp objects.

### BACKGROUND

The removal of objects from tubular vessels within a living body can be difficult. For example, naturally occurring objects that can be difficult to remove include thrombi and clots, and artificial objects that can be difficult to remove include valvular prostheses, closure devices for patent foramen ovale (PFO), atrial septal defect devices, ventricular septal defect devices, stents, and the like. Artificial objects can be especially difficult to remove when those objects expand during placement and/or have sharp edges or protrusions. When removing objects from tubular vessels in living bodies, thin-walled arteries and vessels can be traumatized.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates an embodiment of a large mouth snare device being advanced through the inferior vena cava through a large bore flexible sheath, and a target object (illustrated as a displaced CardioSeal™ device) is shown in the pulmonary artery, in accordance with various embodiments;

FIG. 2 illustrates a snare device being advanced toward a target object in the pulmonary artery, in accordance with various embodiments;

FIG. 3A illustrates a snare device grasping the target object in the pulmonary artery. in accordance with various embodiments;

FIG. 3B illustrates a close-up view of the snare device and target object shown in FIG. 3A, in accordance with various embodiments;

FIG. 4 illustrates a flexible sheath being advanced up the pulmonary artery toward a snare device, in accordance with various embodiments;

FIG. 5 illustrates the deployment of a funnel device when the flexible sheath is withdrawn, in accordance with various embodiments;

FIG. 6A illustrates a snare device and target object being retracted into the mouth of a funnel device and enveloped at least partially therein, in accordance with various embodiments;

FIG. 6B illustrates a close-up view of the snare device and target object being retracted into the mouth of the funnel device shown in FIG. 6A, in accordance with various embodiments;

FIG. 7 illustrates a snare device and target object completely enveloped by a funnel device, in accordance with various embodiments;

FIG. 8 illustrates a funnel device being retracted back into a flexible sheath with snare device and target object completely enveloped therein, in accordance with various embodiments;

FIG. 9 illustrates a funnel device fully retracted back into a flexible sheath with a snare device and target object completely enveloped therein, in accordance with various embodiments;

FIG. 10 illustrates a flexible sheath (which contains a snare device, target object, and funnel device, not shown) being withdrawn into a larger sheath, such as a 24 French sheath, in the inferior vena cava and out of the body, in accordance with various embodiments; and

FIG. 11 illustrates the heart, lungs, pulmonary arteries, inferior vena cava, and associated structures, and shows a loose CardioSeal™ device in the pulmonary artery, an aortic percutaneous valve that has been displaced in the mid aorta, and a stent that has been misplaced in the iliac artery, all of which may be safely removed using the devices and methods disclosed herein, in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “NB” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments disclosed herein provide devices and methods for removing objects, such as thrombi, clots, or man-made objects, from the vasculature without damaging vascular or cardiac structures. In various embodiments, the devices may include a conventional vascular snare device, such as those disclosed in U.S. patent application Ser. No. 12/021,204, which is herein incorporated by reference in its entirety. The snare device may be deployed through a large bore flexible sheath that contains an expandable funnel element at its distal end. In use, once the snare device has been used to capture a desired object, the object may be retracted into the expanded funnel element, which may then partially or completely envelop the captured object and protect the surrounding vascular structures from damage. In some embodiments, the funnel element may then be retracted into the large bore flexible sheath, which may expand to envelop the object and secure it within the device, thus enabling the object to be safely withdrawn from the body without causing damage to the vasculature.

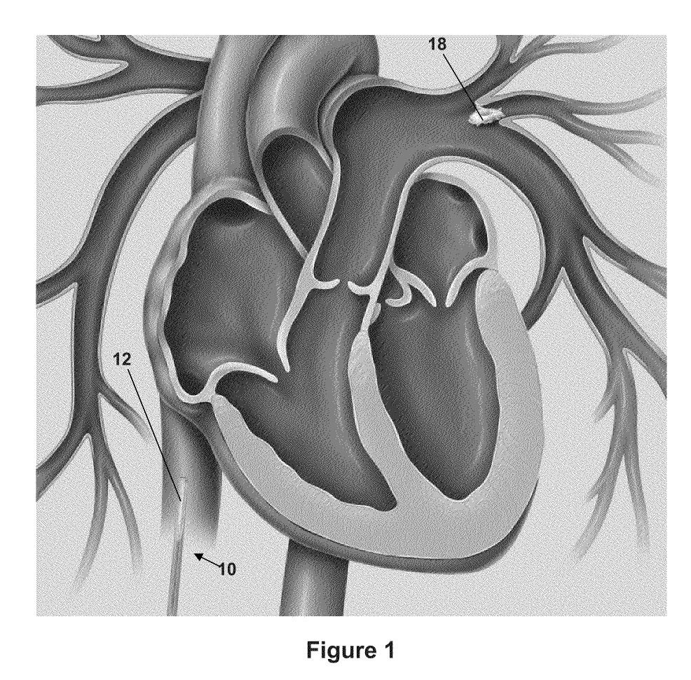

FIG. 1 depicts an embodiment of the large mouth snare device 10 being advanced through the inferior vena cava through a large bore flexible sheath 12 such as a, 18 French sheath. In some embodiments, flexible sheath 12 may be made from a silastic or rubber-like material that may be very malleable, expandable, and/or flexible. Specific, non-limiting examples of suitable materials include polytetrafluoroethylene (PTFE), nylon, plastic, silicone, neoprene, rubber, and expandable urethane. In various embodiments, flexible sheath 12 may be advanced over a guidewire (not shown) such as a 0.014 inch-0.035 inch wire, and may be contained inside a second, larger sheath (not shown) such as a 24 French sheath. Flexible sheath 12 also may contain funnel device 16, which may be advanced along guidewire 14 in the collapsed state along with flexible sheath 12. FIG. 1 also depicts a target object 18 (which is illustrated as a displaced CardioSeal™ device, but may be any naturally-occurring or man-made object) in the pulmonary artery.

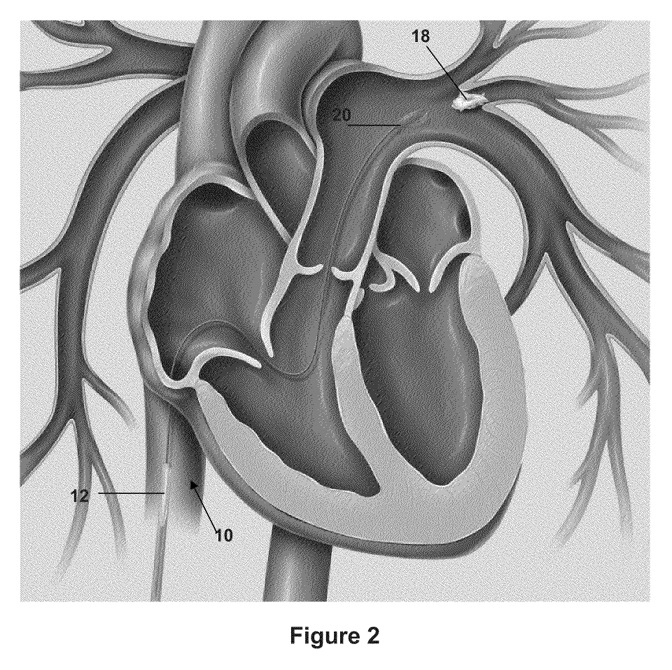

FIG. 2 illustrates a snare device 20 being advanced toward target object 18. Any snare system can be used with the disclosed system, such as the Elite™ manufactured by Radius Inc., the Gooseneck Snare Device manufactured by EV3, those disclosed in U.S. patent application Ser. No. 12/021,204, or any other snare or claw device, although a claw-type snare is depicted. In some embodiments, snare device 20 may be advanced along a guidewire if the guidewire has already been advanced to target object 18. Alternatively, the guidewire may be withdrawn and snare device 20 may be advanced without a guidewire.

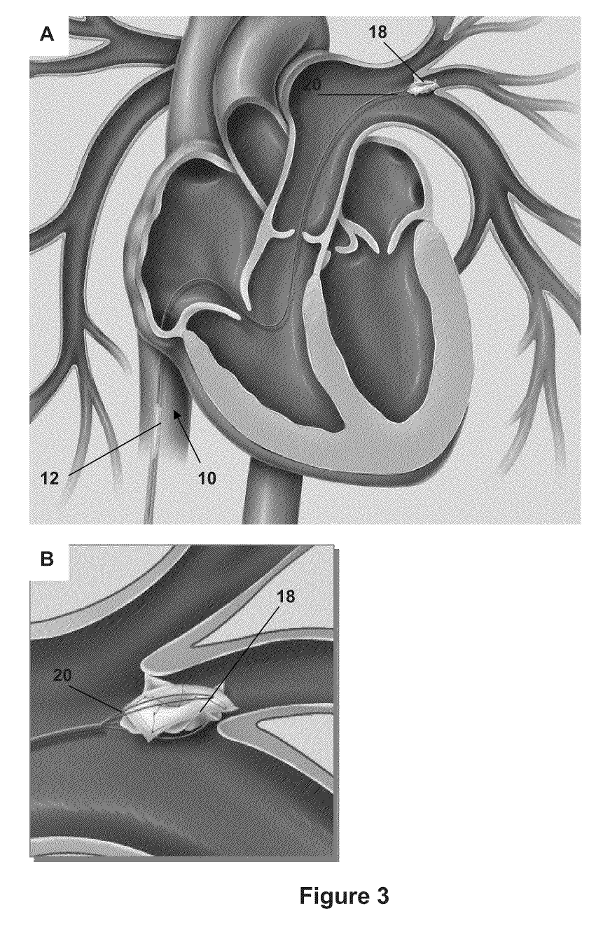

FIG. 3A depicts snare device 20 grasping target object 18 in the pulmonary artery. In some embodiments, target object 18 may have one or more sharp edges or corners, which may cause damage to the vasculature during removal. For example, in addition to naturally occurring objects such as clots, foreign bodies sometimes must be removed from the vasculature. These foreign bodies may include, for example, components of artificial valves, patches used for repairing atrial septal defects (ASDs), or other man-made devices such as embolism-preventing filters, stents, closure devices for patent foramen ovale (PFO), closure devices for ventricular septal defects, and the like. For example, an ASD patch deviceβ may become separated from the cardiac tissue and embolize into the right atrium and/or pulmonary artery. Examples of ASD devices include, for example, the CardioSeal™ device shown in FIG. 3B, the AGA Amplatzer™, the Gore Helix™ device. Other intravascular devices also can be removed with the snare device 20.

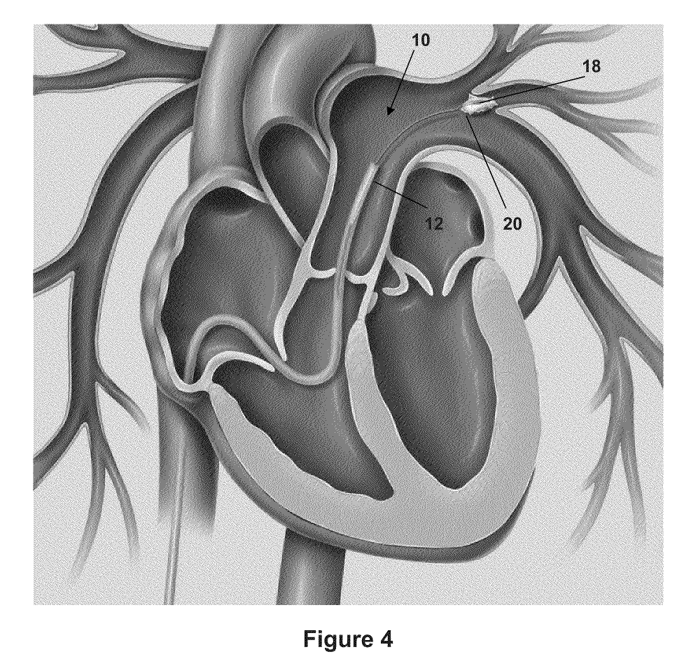

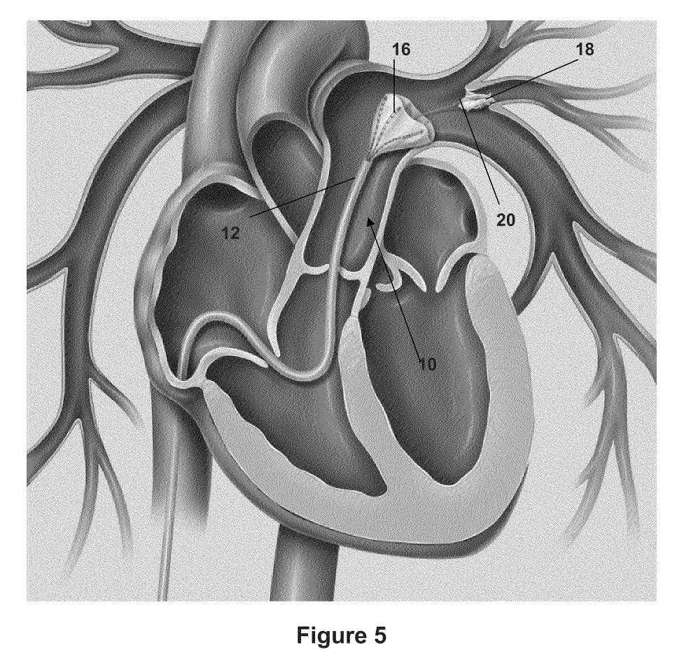

FIG. 4 illustrates flexible sheath 12 being advanced up the pulmonary artery toward snare device 20. FIG. 5 illustrates the deployment of funnel device 16 when flexible sheath 12 is withdrawn. Although funnel device 16 is depicted as having a funnel shape, one of skill in the art will appreciate that the device may have other shapes that may be configured to open and envelop target object 18. For instance, funnel device 16 may comprise one or more overlapping petals, and may have a flower or tulip shape. As used herein, the term “funnel device” refers to any such device having a generally funnel-shaped or flower-shaped configuration, and generally having a closed end and an open end for receiving target object 18. In various embodiments, funnel device 16 may be made from any flexible material, such as fabric, cloth, or mesh material, for example made from PTFE or nylon, or a fine metal mesh such as Nitinol, or any other flexible and expandable material. In some embodiments, funnel device may be biased in the open position such that withdrawing flexible sheath 12 causes funnel device 16 to open. For example, funnel device 16 may include one or more ribs 22 or spring elements to bias it in the open position. Alternatively, funnel device 16 may not be biased in the open direction, but may be positioned such that the flow of blood through the vasculature tends to cause it to open.

In some embodiments, funnel device 16 may be sufficiently porous to allow blood to flow through it even when in an open position. When open, the funnel may have an outside diameter of up to about that of a 50 French catheter (e.g., about 17 mm). In some embodiments, funnel device 16 may have a proximal end and a distal end, and may couple to a hollow wire or catheter at or near the proximal end. In some embodiments, the hollow wire or catheter may pass through funnel device 16 and extend beyond the distal end of funnel device 16. In other embodiments, the hollow wire or catheter may terminate within funnel device, for instance at the proximal end of funnel device 16. In some embodiments, the hollow wire or catheter may have a lumen that is sized to accommodate the diameter of the guidewire.

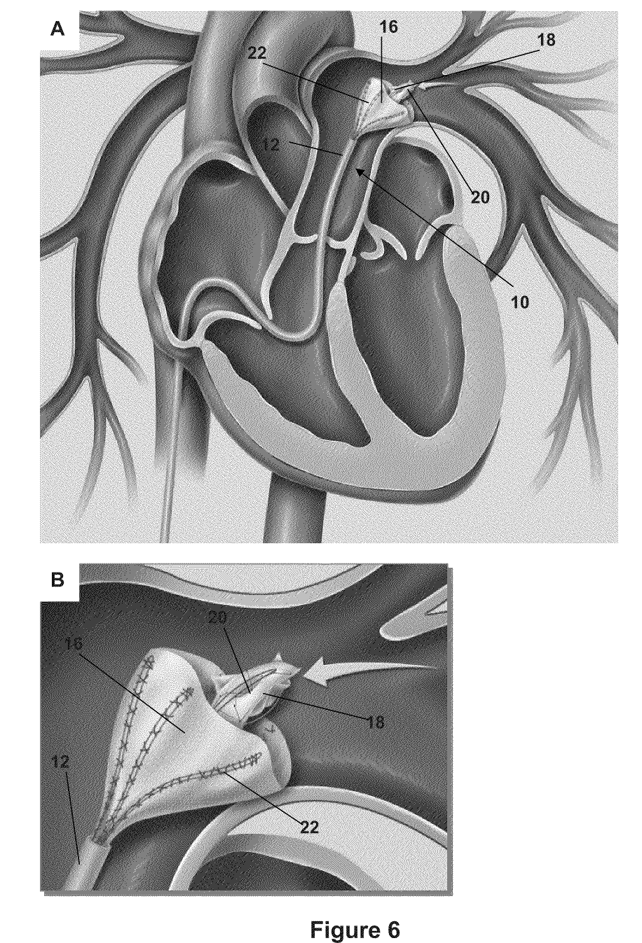

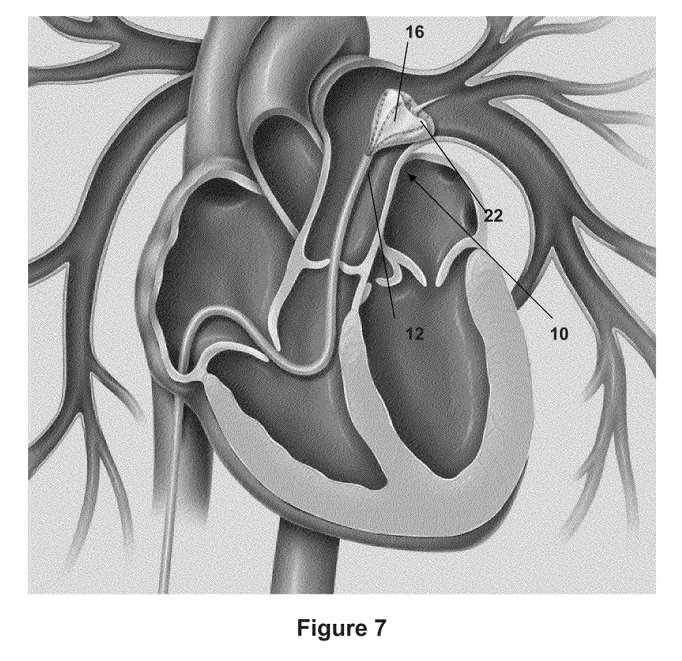

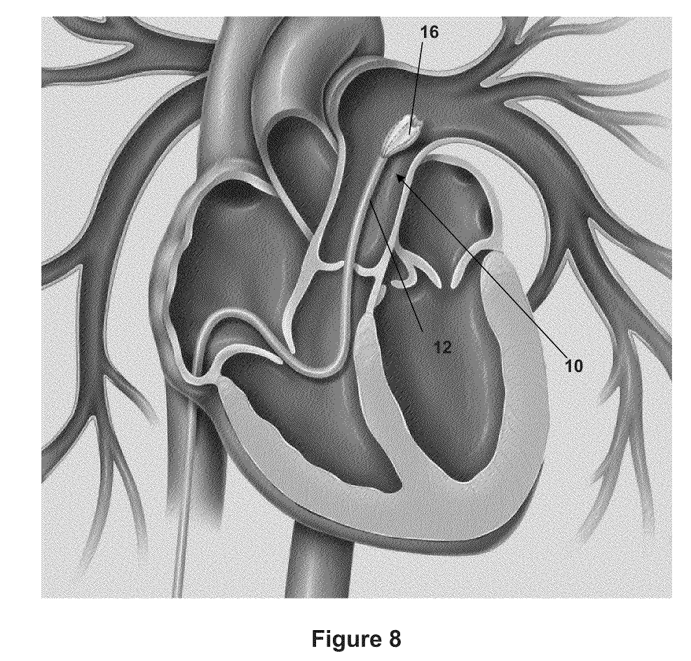

As shown in FIG. 6A, once funnel device 16 is open, snare device 20 and target object 18 may be retracted into the mouth of funnel device 16 and enveloped at least partially therein. In various embodiments, funnel device 16 may be sized to be large enough to partially or completely envelop a large target object 18, as illustrated in FIG. 6B. FIG. 7 illustrates snare device 20 and target object 18 completely enveloped by funnel device 16, and FIG. 8 illustrates funnel device 16 being retracted back into flexible sheath 12 with snare device 20 and target object 18 completely enveloped therein.

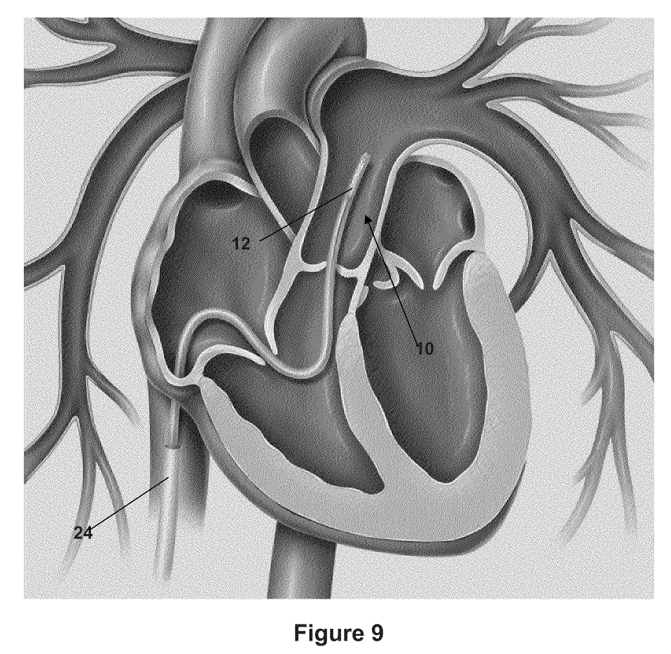

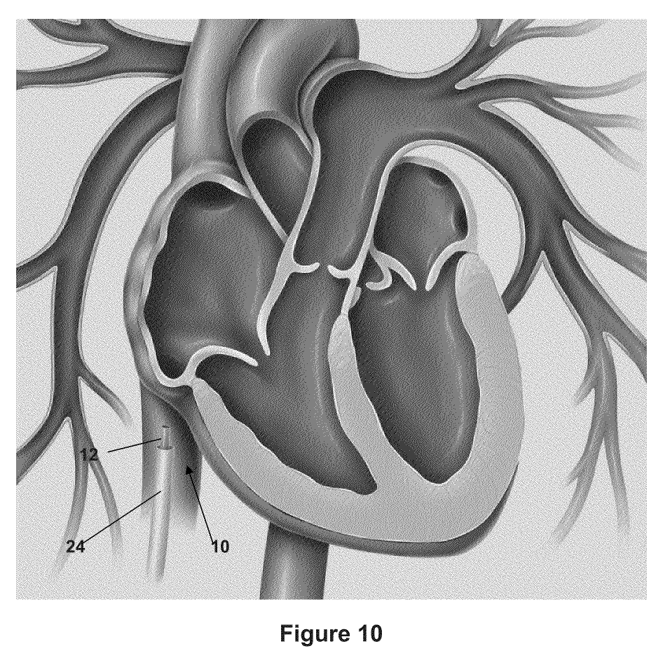

FIG. 9 illustrates funnel device 16 fully retracted back into flexible sheath 12 with snare device 20 and target object 18 completely enveloped therein. Although not shown in FIG. 9, in use, flexible sheath 12 may stretch to accommodate target object 18, such that the outer diameter of flexible sheath 12 may be several times larger in the expanded state than in the non-expanded state, for example to the diameter of a 39 French (−13 mm) or 42 (−14 mm) French sheath. Thus, in various embodiments, funnel device 16 and flexible sheath 12 may fully envelop any sharp edges or corners of target object 18, and may protect the vasculature and cardiac structures from damage while target object 18 is removed from the body. FIG. 10 illustrates flexible sheath 12 (which contains snare device 20, target object 18, and funnel device 16, not shown) being withdrawn into a larger sheath 24, such as a 24 French sheath, in the inferior vena cava and out of the body. The next slide shows the device has been removed.

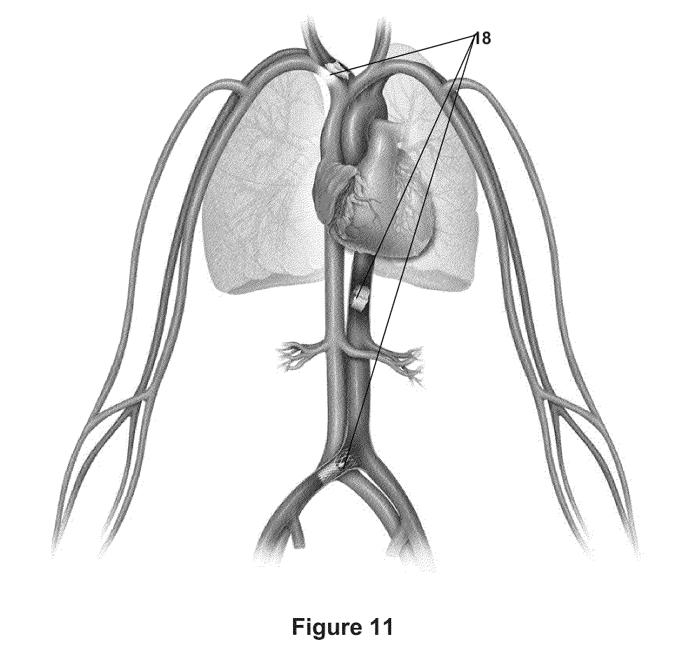

FIG. 11 depicts the heart, lungs, pulmonary arteries, inferior vena cava, and associated structures, and shows a loose CardioSeal™ device in the pulmonary artery, an aortic percutaneous valve that has been displaced in the mid aorta, and a stent that has been misplaced in the iliac artery. All of these are foreign objects that may be safely removed using the devices and methods disclosed herein.

In use, large mouth snare device 10 may be inserted into the vasculature in any conventional way, for example via a femoral vein or artery, and may be manipulated through the vasculature until its distal end is adjacent to target object 18. snare device 20 may then be extended from an inner lumen of the large mouth snare device 10, and may be manipulated such that it grasps target object 18. Such manipulations depend on the type of snare device used, and are well known to those of skill in the art.

Either before or after snare device 20 is deployed, flexible catheter 12 may be retracted slightly, exposing funnel device 16. When exposed, funnel device may then open, either because it is configured to be biased in the open direction or by the force of blood flow against the open mouth of funnel device 16.

Once snare device 20 has grasped target object 18, snare device 20 and target object 18 may be withdrawn partially or completely into open funnel device 16 and may be completely or partially encompassed therein. Funnel device 16, snare device 20, and target object 18 may then be pulled into the distal end of flexible sheath 24, which may expand to envelop all or part of funnel device 16, snare device 20, and target object 18. Once so enveloped, target object 18 may be safely removed from the body by retracting large mouth snare device from the vasculature.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. An apparatus for removing an object from a vessel, the apparatus comprising:
an expandable catheter having a first distal end insertable to a position adjacent to the object within the vessel, a first opening adjacent to the first distal end, and a first lumen in communication with the first opening;
an expandable funnel within the first lumen, the expandable funnel being manipulable between a closed position wherein the expandable funnel is compressed within the first lumen, and an open position, wherein the expandable funnel extends out of the first opening and has a diameter greater than the expandable catheter; and
a snare device extending from the first lumen and configured to capture an object in the vessel and retract the object at least partially into the expandable funnel.

2. The apparatus of claim 1, wherein the expandable catheter is configured to expand to at least partially encompass the expandable funnel, snare device, and object.

3. The apparatus of claim 1 wherein the expandable funnel comprises PFTE, nylon, or Nitinol.

4. The apparatus of claim 1, further comprising a first hollow wire having a second distal end, a second opening and a second lumen in communication with the second opening, the first hollow wire being positioned within the first lumen and coupled to the expandable funnel.

5. The apparatus of claim 4, wherein the first hollow wire is coupled at the second distal end to the funnel.

6. The apparatus of claim 4, further comprising a guidewire configured to pass within the second lumen.

7. The apparatus of claim 6, wherein the guidewire has a diameter of about 0.014-0.035 inches.

8. The apparatus of claim 1, wherein the expandable catheter is a 24 French catheter.

9. The apparatus of claim 1, wherein the expandable catheter comprises a silastic material.

10. The apparatus of claim 1, wherein the expandable catheter is configured to expand to a diameter of about 13-14 mm.

11. The apparatus of claim 1, further comprising a large bore catheter having a third distal end, a third opening adjacent to the third distal end, and a third lumen in communication with the third opening, wherein the large bore catheter is adapted to accommodate the expandable catheter within the third lumen.

12. An apparatus for removing an object from a vessel, the apparatus comprising:
an expandable catheter having a first distal end insertable to a position adjacent to the object within the vessel, a first opening adjacent to the first distal end, and a first lumen in communication with the first opening;
an expandable funnel within the first lumen, the expandable funnel comprising PFTE, nylon, or Nitinol, and being manipulable between a closed position wherein the expandable funnel is compressed within the first lumen, and an open position, wherein the expandable funnel extends out of the first opening and has a diameter greater than the expandable catheter;
a first hollow wire having a second distal end, a second opening and a second lumen in communication with the second opening, the first hollow wire being positioned within the first lumen and coupled to the expandable funnel;
a guidewire configured to pass within the second lumen; and
a snare device extending from the first lumen and configured to capture an object in the vessel and retract the object at least partially into the expandable funnel, wherein the expandable catheter is configured to expand to at least partially encompass the expandable funnel, snare device, and object.

13. The apparatus of claim 12, wherein the guidewire has a diameter of about 0.014-0.035 inches.

14. The apparatus of claim 12, wherein the expandable catheter is a 24 French catheter.

15. The apparatus of claim 12, wherein the expandable catheter is configured to expand to a diameter of about 13-14 mm.

16. The apparatus of claim 12, further comprising a large bore catheter having a third distal end, a third opening adjacent to the third distal end, and a third lumen in communication with the third opening, wherein the large bore catheter is adapted to accommodate the expandable catheter within the third lumen.

17. A method of removing an object from a vessel, the method comprising:
providing an expandable catheter having a first distal end insertable to a position adjacent to the object within the vessel, a first opening adjacent to the first distal end, and a first lumen in communication with the first opening; an expandable funnel within the first lumen, the expandable funnel being manipulable between a closed position wherein the expandable funnel is compressed within the first lumen, and an open position, wherein the expandable funnel extends out of the first opening and has a diameter greater than the expandable catheter; and a snare device extending from the first lumen and configured to capture an object in the vessel and retract the object at least partially into the expandable funnel;
inserting the expandable catheter to a first position wherein the first distal end is adjacent to the object within the vessel;
manipulating the snare device to capture the object;
retracting the snare device and the object into the expandable funnel;
retracting the expandable funnel into the expandable catheter; and
retracting the expandable catheter to remove the expandable catheter, expandable funnel, snare device, and object from the vessel without causing injury to the vessel.

18. The method of claim 17 further comprising the step of withdrawing the expandable sheath sufficiently to expose the expandable funnel.

19. The method of claim 18, wherein the step of withdrawing the expandable sheath sufficiently to expose the expandable funnel is carried out before the step of retracting the snare device and the object into the expandable funnel.

20. The method of claim 18, wherein the step of withdrawing the expandable sheath sufficiently to expose the expandable funnel is carried out before the step of manipulating the snare device to capture the object.
